(19) United States
(12) Patent Application Publication
(10) Pub. No.: US 2015/0366827 A1
(43) Pub. Date: Dec. 24, 2015

(54) COMPOSITIONS AND METHODS FOR MIXING AND APPLYING MIXED OXIDANT COMPOSITIONS FOR DAIRY ANIMAL TREATMENT

(71) Applicant: GEA Farm Technologies, Inc., Naperville, IL (US)

(72) Inventors: Solomon Brown, Romeoville, IL (US); Charles Gradle, Oak Park, IL (US); Randall Richards, Dallas, TX (US)

(21) Appl. No.: 14/752,162

(22) Filed: Jun. 26, 2015

Related U.S. Application Data
(63) Continuation-in-part of application No. 14/696,289, filed on Apr. 24, 2015.
Application No. 14/696,289 is a division of application No. 12/925,846, filed on Oct. 29, 2010, now abandoned.
(60) Provisional application No. 62/017,940, filed on Jun. 27, 2014.
(60) Provisional application No. 62/087,872, filed on Dec. 5, 2014.
(60) Provisional application No. 61/280,163, filed on Oct. 30, 2009.

(51) Int. Cl. A61K 31/185; A61K 47/14; A61K 31/13; A61K 47/24

(52) U.S. Cl. CPC A61K 31/185; A61K 47/24; A61K 47/14; A61K 31/13

(57) ABSTRACT

A method for mixing dairy animal teat dip from water and additives. The method includes a mixing manifold into which the water and additives are fed and mixed in a controlled manner. Mixed teat dip is automatically quality tested and monitored to provide data for controlling quantities of water and additives being fed to the manifold.

### CROSS REFERENCE TO RELATED APPLICATIONS

This application claims the benefit of Provisional Application 62/087,872, filed Dec. 5, 2014, and Provisional Application 62/017,940, filed Jun. 27, 2014, and is a continuation-in-part of U.S. application Ser. No. 14/696,289, filed Apr. 24, 2015, a divisional of U.S. application Ser. No. 12/925,846 filed Oct. 29, 2010, and claims the benefit of Provisional Application 61/280,163 filed Oct. 30, 2009, the disclosures of which are incorporated by reference herein.

### FIELD AND BACKGROUND OF THE INVENTION

The present invention relates to methods and apparatus for mixing dairy animal treatment chemicals and, in particular, for mixing dairy animal teat dip compositions having a mixed oxidant and an additive to enhance the usefulness of the composition.

In dairy harvesting facilities, dairy animals are commonly treated with antimicrobial teat dips (“teat sanitizers”) before and/or after milking. Teat sanitizers reduce or eliminate bacteria or other microorganisms that can cause an infection and inflammation of the mammary gland of milk-producing animals such as cows, goats, sheep, and buffalo. This infection, known as mastitis, causes a significant loss in income to dairy farmers worldwide. Some figures in the United States from 1993, for example, state losses in excess of two billion dollars, which includes not only loss in actual milk production, but loss of quality bonuses/premiums, treatment costs, costs related to culling and replacement of animals, and such indirect costs as increased training costs for employees, increased monitoring costs, associated medical costs related to mastitis in addition to animal welfare considerations (Oorsigartikel, A Review of the Factors Affecting the Costs of Bovine Mastitis; K R Petrovskia*, M Trajcevb and G Buneskib 0038-2809 Jl S.Afr.vet.Ass. (2006) 77(2): 52-60; and Jones, G. M.; Bailey, T. L. “Understanding the Basics of Mastitis”. Virginia Cooperative Extension). Teat sanitizers have been shown in many field trials on dairy farms to be effective in preventing mastitis, both by killing microorganisms that may be present before milking machine attachment (environmental pathogens) and after the milking of the animal is concluded (contagious pathogens).

Most teat dips are premixed at a chemical mixing plant and shipped to dairies as ready-to-use products. Premixed teat dips often include a large percentage of water that adds bulk and weight to the product and requires substantial shipping and storage costs. Further, some teat dips are unstable and have short shelf lives. Unstable teat dips are sold and shipped with unmixed components that are mixed in batches at a dairy facility or a dairy dealership near the dairy for use while it is still effective.

Stable compositions of mixtures of surfactants and oxidizing materials that produce efficacious teat sanitizers have been described for iodine (U.S. Pat. No. 5,503,838), peracetic acid (U.S. Pat. No. 8,034,759), and peroxide (U.S. Pat. No. 5,139,788). Additionally, stable hypochlorite compositions containing surfactants have been described (U.S. Patent Application Publication No. 2006/0263240).

Another type of oxidizer useful in teat dips is a mixed oxidant. Mixed oxidants can be inexpensively produced with an automated system using an electrolysis process that supplies an electrical current through a salt solution and collects the resulting oxidant mixture. This process has been described in U.S. Pat. No. 7,008,523. The nature of the mixed oxidant solution that is produced by this electrolysis process has been described as containing multiple oxidizing species comprising hypochlorite, peroxide, chlorine dioxide, chlorine radicals, and oxygen radicals. The mixed oxidant solutions have been reported to be more reactive than ordinary oxidants.

Oxidizing materials that include iodine, peroxides, chlorine dioxide, peracetic acid, and hypochlorite have been combined with surfactants to produce stable solutions with good antimicrobial properties, but when a surfactant, colorant, or other additive is added to a mixed oxidant, the mixture can become unstable and have diminished teat sanitizing properties and shelf life.

Some of the advantages of using surfactants in teat sanitizers include better cleaning of the teat skin and better penetration of the germicidal component(s) into the skin surface of the teat, comprising folds and crevices that may be present where harmful microorganisms may be sequestered.

Mixing systems have been described for use in dairy facilities to make cleaning and hygiene products for use on dairy cows. Some of these systems require various scale, meter and pump configurations to accomplish accurate measuring and mixing of chemicals. Flow measurement and gravimetric measurement are two of the primary measuring methods that have been used to combine individual components into a chemical product. Gravimetric means to blend products require precise weighing of chemical components on one or more scales before the components are mixed.

Another mixing system requires a vacuum for moving chemicals to a mixing vessel. Vacuum systems can be unreliable and imprecise because no monitoring of the flow rate is performed and delivery time for the ingredients is used to determine the amount of each component added at a mixing station.

There are also chemical dilution and dosing systems that use two flow meters, one on a single ingredient entering a mixing manifold and a second meter measuring flow of mixed material exiting the manifold. A disadvantage of this type of system is that it cannot measure more than one ingredient at a time as it enters a mixing manifold. Additionally, such systems typically use air to cause the ingredient to transfer into a mixing manifold, which can be unreliable and difficult to control.

Prior chemical mixing systems suffer from an inherent non-uniform mixing of the finished chemical product and may require an additional mixing step to make a homogenous finished chemical product.

For these reasons, dairy animal treatment chemicals, particularly teat dips, are generally mixed by a manufacturer at a primary chemical mixing facility to ensure complete mixing and teat dip quality. The premixed teat dips are usually shipped to dealers in dairy producing regions, and then sold to dairy harvesting facilities. Teat dips with short shelf lives are not sold this way because they lose efficacy during multiple stages of shipping. Teat dips that are chemically stable are sold and distributed this way, but as stated above, shipping and packaging costs are a substantial portion of a teat dip's volume and price.

Thus, there is a need for stable teat dip compositions that include a mixed oxidant and an additive, such as a surfactant, which can be mixed near a point of use, such as a dairy.

### SUMMARY OF THE INVENTION

The present invention is directed to mixed oxidant compositions and methods for mixing a mixed oxidant with additives to enhance the effectiveness of the compositions for use on dairy animals. In accordance with the present invention, there is provided a method for sanitizing a dairy animal teat after milking by performing the steps of: mixing a surfactant and a mixed oxidant to produce a composition that is stable and is a teat sanitizer; and applying the composition to the dairy animal teat.

The surfactant can be selected from the group of surfactants including quaternary ammonium compounds, sulfates, sulfonates and amine oxides, and combinations thereof. The method can also include the step of mixing a stabilizer in the teat dip composition.

There is also a provided in accordance with the present invention, a method to increase the antimicrobial efficacy of a mixed oxidant by combining the mixed oxidant with a surfactant in an aqueous solution that produces a stable composition. The method can also include the step of mixing a colorant with the mixed oxidant, surfactant, and an aqueous solution shortly before the teat dip is used.

Another embodiment of the present invention is a method for sanitizing dairy animal teats, with the steps of combining a rheological modifier with a mixed oxidant and applying the mixture to the teat of the dairy animal.

Another embodiment of the present invention is a method for sanitizing dairy animal teats by combining a secondary antimicrobial agent with a mixed oxidant and applying the mixture to the dairy animal teat after milking the dairy animal.

A stable ready to use teat sanitizer composition in accordance with the present invention includes from about 1% to about 99% of water by weight of composition, from about 0.1% to about 80.0% of antimicrobial agent by weight of composition, from about 0.01% to about 10.0% of thickener by weight of composition, from about 0.001% to 10.0% of colorant by weight of composition, and from about 1000 ppm to about 8000 ppm of a mixed oxidant.

The composition thickener can be selected from the group of thickeners consisting of: polyacrylates, acrylates, carbomers, acrylate containing polymers, acrylate containing copolymers, and combinations thereof.

The present invention is also directed to a method for sanitizing dairy animal teats, the method including the steps of: mixing a concentrate including water, an antimicrobial agent, a thickener, and a colorant, and diluting the concentrate with water and a mixed oxidant to make a teat sanitizer including from about 1% to about 99% of water by weight of composition, from about 0.1% to about 80% of antimicrobial agent by weight of composition, from about 0.01% to about 10% of thickener by weight of composition, from about 0.001% to about 10.0% of colorant by weight of composition, and from about 0.01% to about 0.8% of mixed oxidant by weight of composition.

A dilutable dairy animal teat sanitizer composition in accordance with the present invention includes: from about 1% to about 95% of water by weight of composition, from about 0.5% to about 80% of antimicrobial agent by weight of composition, from about 0.05% to about 25% of thickener by weight of composition, and from about 0.005% to about 30% of colorant by weight of composition, whereby the dilutable dairy animal teat sanitizer composition can be diluted with water and a mixed oxidant resulting in a ready-to-use composition including, from about 1% to about 99% of water by weight of composition, from about 0.1% to about 80% of antimicrobial agent by weight of composition, from about 0.01% to about 10% of thickener by weight of composition, from about 0.001% to about 10.0% of colorant by weight of composition, and from about 0.01% to about 0.8% of mixed oxidant by weight of composition.

A stable ready-to-use teat sanitizer composition in accordance with the present invention includes: from about 1% to about 99% of water by weight of composition, from about 0.1% to about 80.0% of a secondary antimicrobial agent by weight of composition, from about 0.01% to about 10.0% of thickener by weight of composition, from about 0.001% to about 10.0% of Pigment Green 7 or of Phthalocyanine Blue BN by weight of composition, and from about 1000 ppm to about 8000 ppm of mixed oxidant.

A substantially stable dairy animal teat sanitizer composition in accordance with the present invention includes: from about 1% to about 90% of water by weight of composition, from about 0.1% to about 80.0% of antimicrobial agent by weight of composition, from about 0.05% to about 50.0% of thickener by weight of composition, and from about 0.005% to about 40.0% of Pigment Green 7 or of Phthalocyanine Blue BN by weight of composition.

The composition can be diluted with water or a mixed oxidant at a ratio of about 1:1 composition to diluent to a ratio of about 1:1,000 solution to diluent and in some embodiments diluted in a ratio range of about 1:2 composition to diluent to about 1:250 solution to diluent. In still another embodiment, the dairy animal teat sanitizer composition is diluted with water or a mixed oxidant at a ratio of about 1:5 composition to diluent to a ratio of about 1:50 composition to diluent.

In another embodiment, a concentrated dairy animal teat sanitizer composition is provided that includes: from about 1% to about 90% of water by weight of composition, from about 0.1% to about 80.0% of antimicrobial agent by weight of composition, from about 0.05% to about 50.0% of thickener by weight of composition, from about 0.005% to about 40.0% of first colorant by weight of composition, and from about 0.005% to about 40.0% of second colorant by weight of composition, wherein the first colorant is degradable in the presence of a mixed oxidant and the second colorant is not degradable in the presence of a mixed oxidant.

The concentrated dairy animal teat sanitizer composition first colorant can include Pigment Violet 23, FD&C Blue 1, Liquitint Blue HP, Liquitint Red ST, Liquitint Pink AL, Yellow #5, Yellow #6, Methylene Blue, FD&C Red #40, Aquamarine Blue, and combinations thereof. The concentrated dairy animal teat sanitizer composition second colorant can include Pigment Green 7, Phthalocyanine Blue BN Pigment Violet 23.

The present invention is also directed to a stable dairy animal teat sanitizer composition including: from about 1% to about 99% water by weight of composition, from about 500 ppm to about 10,000 ppm of a mixed oxidant and from about 0.0001% to about 10% of a phthalocyanine pigment by weight of composition. The composition can also include from about 0.1% to about 50% by weight of composition of a stable emollient. The emollient can be selected from a group consisting essentially of: esters, glycol esters, glycol diesters, PEG esters, amine oxide, and combinations thereof.

Further, the phthalocyanine pigment can be selected from the group consisting essentially of: Direct Blue 86, Direct Blue 199, C.I. Pigment Blue 15, C.I. Pigment Green 36, C.I. Pigment Green 7, C.I. Pigment Violet 23, and Phthalocyanine Blue BN, and combinations thereof. The composition can also contain from about 0.05% to about 50% of a viscosity modifier by weight of composition. The viscosity modifier can include: PVP, polyvinyl alcohol, polyacrylic acids, carbomers. cross-linked polyacrylic acids, benzene homopolymers, cosolvent polymers, hydrophobically modified copolymers, acrylate/alkyl acrylate cross polymers, polyethylene glycol and combinations thereof.

The stable dairy animal teat sanitizer composition can also contain from about 0.1% to about 80% by weight of composition of a secondary antimicrobial. The secondary antimicrobial can be selected from a group consisting essentially of: chlorhexidine, biguaunide, fatty acids, lactic acid, anionic surfactants, amine oxides, bronopol, amines, nisin, glycerol monolaurate, quaternary ammonium compounds, laurel amine, DDBSA, Alkyl Dimethyl amine Oxide, and combinations thereof.

In addition to the methods and compositions described above, the present invention eliminates the deficiencies of prior mixing and dispensing systems by creating a mixed oxidant and mixing it with chemical teat dip additives and water at a dairy facility or a regional dealer's facility, using a mixing manifold. The invention includes various data feedback mechanisms to determine whether correct mixing ratios, flow rates, and ingredient quantities are being used, so that appropriate adjustments can be made by a system controller during the mixing process.

Measuring, feeding, and mixing rates for water and additives are monitored and controlled using preset algorithms, meters, pumps, and sensors to obtain a finished teat dip product that has properties within desired tolerances.

The present invention can also be located at or near a point of use to accurately mix ingredients in desired ratios, and includes, a mixing manifold for receiving mixed oxidants, water and at least one additive for mixing. Quantities of water and additives are monitored by meters that can be located between the fluid sources and the mixing manifold. For optimum mixing, a controller can energize and de-energize ingredient pumps so that precise quantities of ingredients are supplied to the mixing manifold. This can be done using a preset algorithm wherein the pumps preferably begin and end pumping each ingredient at substantially the same time and at a substantially predetermined rate for each ingredient. The rate of pumping can be controlled by the controller using data feedback from the meter and/or quality control sensors that detect mixed teat dip quality or other devices that monitor ingredients, the system components or ambient conditions.

Teat dip produced from the present invention can be used without further mixing, storage or processing. Alternately, the mixed chemical product can be dispensed into a storage container without further processing for later use in spraying, dipping, cleaning, rinsing or otherwise utilizing the teat dip. Mixed teat dip can be stored in a tank, drum, tote or any other container with which the teat dip product is compatible. The mixed oxidant can also be mixed and stored as a concentrate for further processing later in time.

The present invention is also directed to a method for mixing teat dip ingredients using a mixing manifold for mixing water from a source at or near a point of use, and additives such as antimicrobial agents, surfactants, emollients, thickeners, fragrances, acids, bases, solvents, and alcohols, for example. The additives are obtained by a dairy chemical dealer or a dairy harvesting facility and then mixed with a local source of water or another type of carrier.

Water is fed to the mixing manifold, and meters are used for monitoring and/or controlling the quantity of water, obtaining water flow data and transmitting that data to a controller. The water flow data is used by the controller to activate and control pumps to deliver at least one additive to the mixing manifold in a predetermined proportion to the water. Additive rate data is obtained by meters or other sensors and transmitted to the controller for comparison to a predetermined additive quantity and the additive data can then be used by the controller to determine whether the additive quantity being fed to the mixing manifold is within a predetermined range or if a pump or valve should be activated to increase or decrease additive flow quantities.

The method further includes the steps of mixing the additive, mixed oxidant, and water in the mixing manifold to obtain a substantially mixed teat dip, obtaining quality control data about the mixed teat dip using a quality control sensor, transmitting the quality control data to the controller, comparing the quality control data in the controller to predetermined quality control data, and determining whether the mixed teat dip is within predetermined quality control parameters. If necessary, the controller can then adjust any of the system components to bring the mixed teat dip within the quality control parameters.

These and other features and advantages of various embodiments of systems and methods according to this invention are described in, or are apparent from, the following detailed description of various exemplary embodiments of various devices, structures, and/or methods according to the present disclosure.

### BRIEF DESCRIPTION OF THE DRAWINGS

FIG. 1 is a schematic view of a teat dip mixing system in accordance with the present invention.

FIG. 2 is a graph depicting temperature of an additive versus an amount dispensed, in accordance with the present invention.

### DETAILED DESCRIPTION OF THE INVENTION

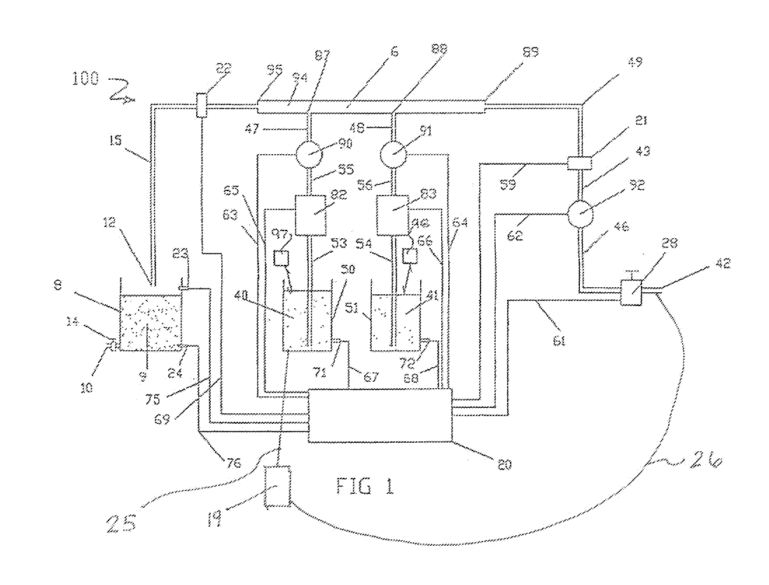

A fluid mixing system 100 of the present invention is shown in FIG. 1, for mixing and dispensing chemical teat dip product 9. The system includes a controller 20, an electrolyzer 19, a number of additive storage tanks 50 and 51, a water supply 42, a mixing manifold 6, and a mixed teat dip storage tank 8. The additive storage tanks 50 and 51 store teat dip ingredients referred to herein as additives. Additives can be chemical concentrates, mixed oxidants, or any other teat dip ingredient, and there can be any number of additives and additive tanks. Some of the additives can be generated by the electrolyzer 19, and transferred to an additive tank 50, for example, via a conduit 25, or be premixed with each other and/or a carrier. Nonetheless, the additive should still be mixed with a substantial amount of water, other carrier or solvent to obtain a usable teat dip.

The additive storage tanks 50, and 51 and water supply 42 are in fluid communication with the mixing manifold 6 via conduits 53, 54, 55, 56, 47, 48, 42, 43, 46, and 49 to feed water and additives to the mixing manifold 6. The mixing manifold 6 has an outlet 95 in communication with the teat dip storage tank 8 via a conduit 15. Water is described in most of the examples herein as a carrier for the additives, but other carriers or solvents can be used as well.

Additives 40 and 41 in the additive storage tanks 50 and 51 are fed to the mixing manifold 6 with pumps 82 and 83, respectively, and the pumps 82 and 83 are disposed in or between respective conduits 53, 55, 47, 54, 56, and 48. To ensure that accurate quantities of additives are pumped to the mixing manifold 6, meters 90 and 91 are used upstream or downstream of each pump 82 and 83, respectively. The meters 90 and 91 preferably measure mass or volume so that an accurate quantity of additive flows to the mixing manifold 6. The meters 90 and 91 are more accurate than relying on pump operation time, for example. The meters 90 and 91 can measure volume of the additive being pumped, but other properties comprising mass or flow rate can be measured.

Water is provided from any suitable local source and may or may not need a separate pump. The water is typically the largest portion of a teat dip mixed in the present invention, so it is obtained from a local source such as a municipal water supply or a well, thereby saving transportation and packaging costs for the water component. The water can be treated, if necessary, prior to being fed to the mixing manifold 6. For example, the water can be softened, sanitized, temperature adjusted, or modified in any desirable way so that the resulting mixed teat dip can have desired properties. One such treatment can include mixing the water with mixed oxidant from the electrolyzer 19. A water valve 28 and a water meter 92 are preferably used to measure and allow a precise quantity of water to flow to the mixing manifold 6. The water valve 28 and the water meter 92 are particularly desirable when no pump is used for the water. The water valve 28 can be any type of valve and it can include a regulator that controls water flow rate and/or pressure. The water valve 28 controls water flow rates and/or pressure so that additives can be added at rates and/or pressures that correspond to the regulated water flow. As used herein, “water valve” includes any type of flow regulator or device that controls the flow of water.

The additive pumps 82 and 83, and water inlet valve 28 are activated and controlled by the controller 20 based on predetermined quantities for the ingredients in the final mixed teat dip. The additive meters 90, 91 and the water meter 92 measure additives and water being pumped into the manifold 6, and transmit corresponding data to the controller 20 that is then used by the controller 20 for controlling pumps and valves throughout the system 100 and to ensure accurate chemical concentrations are used to mix teat dip.

The controller 20 can be any suitable programmable device that is either preprogrammed, programmable by an operator, programmed and re-programmed by a closed-loop logic system or preferably all of these are available on a single controller 20. Various types of operator interfaces can be included, such as keyboards, touch displays, keypads, switches, visual displays, audible sound generators, and others.

The controller 20 preferably uses Central Processing Unit (“CPU”) or other programmable device such as a Printed Circuit Board (“PCB”) to control the pumps, valves, and meters so that proper ratios, temperatures, and properties of water and additives are delivered to the mixing manifold 6. The starting formulation for each desired teat dip product is preferably programmed into the controller 20 to control the amount and/or rate at which each ingredient enters the mixing manifold 6. The controller 20 preferably starts the pumps 82 and 83 for each additive and opens the water valve 28 at substantially the same time. In one embodiment of the present invention the controller 20 activates and responds to pumps, valves and meters to control the flow rate of each additive to correspond to the amount and properties of water called for in the formula. To do so, the controller 20 can include computer code of a mixing algorithm, which preferably can be modified manually by an operator or modified automatically based on data received from any of the system's 100 components.

In the illustrated embodiment, three separate ingredients are supplied to the mixing manifold 6. The two additives 40 and 41 stored in the additive storage tanks 50 and 51 are individually fed to the mixing manifold 6 by their respective pumps 82 and 83 and conduits 55 and 56. Electronic signals from the controller 20 activate and control the pumps 82 and 83 through electrical connections 65 and 66, but wireless technology can also be used. The additives 40 and 41 preferably pass through the additive meters 90 and 91 after passing through the pumps 82 and 83, but the additive meters 90 and 91 can be positioned before the pumps 82 and 83. The additive meters 90 and 91 generate and send signals representing flow quantity or other measured property through the electrical connections 63 and 64 to the controller 20. The conduits 47 and 48 are of any suitable size, shape, and material and connect to the mixing manifold 6 through inlets 87 and 88. As used here, “additive tanks” includes any suitable containers, such as tanks, barrels, totes, bottles or boxes. The additive tanks 50 and 51 can be filled with additives at the mixing location or they could be filled at and transported from a central chemical supply plant for convenience.

It is also possible and may be desirable to control temperatures of the additives 40 and 41 with one or more heat exchangers 97 and 98. Suitable heat exchangers include heating pads or belts that are wrapped around or placed under additive storage tanks 50 and 51. Alternately or additionally, a heat exchanger can be disposed inside the additive storage tanks 50 and 51 Preferably, the heat exchanger is chemically and physically compatible with the additive. In addition, a heat exchanger (not illustrated) can be used to control water temperature. Additive tank pressure can also be controlled to cause or optimize additive flow toward the mixing manifold 6.

The additives 40 and 41 to be mixed with water can be any desired component of a teat dip, comprising individual chemicals, concentrates, solutions, suspensions, emulsions, solvents or combinations thereof.

One preferred type of additive is a mixed oxidant. A mixed oxidant is produced by supplying an electrical current through a salt water solution in an electrolyzer 19, for example. Any suitable salt can be used, including sodium chloride, potassium chloride or any other suitable salt of chloride or combinations thereof. The end product of this electrolysis process can include many types of oxidizers and usually results in a mixed oxidant solution. The mixed oxidant solutions can include chlorine dioxide, hydrogen peroxide, hypochlorous acid, hypochlorite, and radicals of oxygen, ozone, and other oxidizing species. While the exact composition of the mixed oxidant produced during the electrolysis process is dependent on many factors, the resulting mixed oxidant will have substantial oxidizing capacity and antimicrobial activity. Many of the compositions of the present invention contain a mixed oxidant of this type. Further, the electrolyzer 19 can provide a mixed oxidant to the water valve 28 via the conduit 26 as a water treatment in the event the local water source requires such treatment. Further, the electrolyzer 19 can provide mixed oxidant to the water valve 28 for feeding to the mixing manifold 16 with, or in place of, water.

The activity of a mixed oxidant system is preferably measured with an iodometric titration by the addition of excess iodide and acid and titration of the resulting iodine (in the form of triiodide) with sodium thiosulfate. This method can measure the various oxidants in the mixture, comprising chlorine.

A teat dip composition is defined as stable when the activity of the mixed oxidant decreases by less than about 25% over a 12 hour to 72 hour period. The Tables 1 through 3, and 5 below show the stability of mixed oxidants when exposed to various additives for 12 hours to 96 hours. Table 4 shows the stability of various colorants when exposed to a mixed oxide solution. In each Table 1 through 5, the beginning mixed oxidant titrated at between 3800 ppm to 4100 ppm measured as available chlorine.

TABLE 1
Loss of Mixed Oxidant Activity (as measured by thiosulfate
titration) for various emollients.

| | % Loss of mixed oxidant activity after |
|---|---|
| Emollient | 24 hours at (23° C.) exposure to mixed oxidant |
| Propylene glycol | 71 |
| Glycerin | 97 |
| Ethoxylated lanolin | 95 |
| Sorbitol | 78 |
| Octyl stearate | 11 |
| Methyl glucose ether | 61 |
| Diisostearyl Adipate | 13 |
| Dioctyldodecyl | 12 |
| C12-15 Alkyl | 11 |
| Propylene Glycol | 7 |
| Isopropyl Isostearate | 11 |

It may also be desirable to add emollients to a mixed oxidant composition to improve dairy animal teat conditions. Suitable emollients can be selected from the group consisting of esters, glycol esters, glycol diesters, PEG esters, amine oxide, and combinations thereof.

Preferred emollient and mixed oxidant compositions are relatively stable, so that the loss of mixed oxdidant is no more than about 20% within 24 hours of mixing with an emollient. In one embodiment of the present invention, fatty acid esters are included in the composition to provide emoliency. In a more preferred embodiment, methyl, ethyl, propyl or, benzyl fatty acid esters are included in the composition to provide emoliency.

TABLE 2
Loss of Mixed Oxidant Activity (as measured by thiosulfate
titration) for various surface active materials.

| | % Loss of mixed oxidant | # Days exposure to |
|---|---|---|
| Surfactant | activity at (23° C.) | mixed oxidant |
| Maquat MQ 2525 | 35 | 2 |
| Bardac 2280 | 25 | 2 |
| Lauryl amine oxide | 14 | 4 |
| Nonylphenol ethoxylate | 55 | 2 |
| Cocamido propyl betaine | 56 | 1 |

Suitable surfactants may be added to the composition. As seen in tables 2 and 5, some surfactants produce a stable composition. Suitable surfactants may be selected from the group comprised of amine oxides, sulfates, sulfonates, and combinations thereof

TABLE 3
Loss of Mixed Oxidant Activity (as measured by thiosulfate
titration) for various chelation materials.

| | % Loss of mixed oxidant | # Days exposure to mixed |
|---|---|---|
| Chelator | activity at (23° C.) | oxidant |
| Na | 3.5 | 2 |
| Acusol 445 | 3.1 | 2 |
| EDTA 100 | 94 | 0.1 |
| Sodium Gluconate | 90 | 0.1 |

A chelator may be added to the present invention. The chelator must be compatible with the mixed oxidant. Suitable chelators that are stable include sodium Tripolyphosphate and acrylic acid homopolymers Acusol 445.

Colorants are added to teat dips to provide a visual indicator that a teat dip was applied to a diary animal teat. Some traditional colorants, however, become ineffective when introduced to a mixed oxidant, as Table 4 below shows.

TABLE 4
Loss of color for various colorants.

| | Color intensity on teat after 1 day exposure |
|---|---|
| Colorant | to 4000 ppm mixed oxidant |
| FD&C Blue 1 | Clear |
| Liquitint Blue HP | Clear |
| Liquitint Red ST | Clear |
| Liquitint Pink AL | Clear |
| Yellow #6 | Clear |
| Yellow #5 | Clear |
| Methylene Blue | Clear |
| FD&C Red #40 | Clear |
| Aquamarine Blue | Clear |

Preferably, a stable colorant is added to the teat sanitizer composition, but the colorant must be visible on the teat at the Ready-to-Use concentration. Some colorants are unacceptable because they are stable in their color in solution, but they are not visible on the teat after an exposure to the mixed oxidant for a period of time. An example of this behavior is Yellow #5, which still has some color in the composition after exposure to the mixed oxidant, but the visibility of the dye, once applied to a dairy animal teat is unacceptable.

Preferably, stable pigments and dyes are used to provide a color to the teat sanitizer. In one embodiment, the pigment is suspended in a teat sanitizer containing a rheology modifier that suspends a colorant. In one preferred embodiment, a colorant is introduced into the teat sanitizer just prior to the teat dip being used after milking. This approach allows the use of a less stable colorant, but requires a timely mixing process.

In one embodiment of the present invention, a pigment is used to provide color to the teat sanitizer. Suitable pigments are chosen from the phthalocyanine class of pigments comprising Direct Blue 86, Direct Blue 199, C.I. Pigment Blue 15, C.I. Pigment Green 36, C.I. Pigment Green 7, C.I. Pigment Violet 23, and Phthalocyanine Blue BN, and combinations thereof. An additional chemical component may be added to the teat sanitizer to help maintain the phthalocyanine pigment(s) in solution. In one embodiment a rheology modifier (thickener) is added to the teat sanitizer to help maintain the pigment in solution.

Further, a thickener can be added to the present invention in some preferred embodiments. Suitable thickeners can be selected from the group consisting of: gums, polyvinyl alcohol, polyvinylpyrrolidone (PVP), polysaccharides, surfactants, polymers, magnesium aluminum silicates, silica, cellulose ethers, clays, methyl cellulose, carboxymethylcellulose (CMC), polyethylene glycol, alginates, and combinations thereof. In a preferred embodiment, the thickener exhibits pseudoplastic, sheer thinning or thixotropic rheology. Suitable gums that exhibit pseudoplastic rheology include: xanthan gum, guar gum, gum tragacanth, polysaccharides, modified guar gum, locust bean gum, and modified polysaccharides. Suitable polymers that exhibit pseudoplastic rheology include polyvinylpyrrolidone (“PVP”), polyvinyl alcohol, polyacrylic acids, carbomers, cross-linked polyacrylic acids, neutralized polyacrylic acids, neutralized cross-linked polyacrylic acids, benzene homopolymers, cosolvent polymers, hydrophobically modified copolymers, acrylate/alkyl acrylate cross polymers, and combinations thereof. In a preferred embodiment, of the invention acrylates, carbomers, and acrylate containing polymers and copolymers and modified polymers and combinations thereof may be used to thicken compositions of the present invention.

As table 5 below shows, adding an additional or “secondary” antimicrobial to a mixed oxidant can destabilize the mixed oxidant and actually decrease the efficacy of the mixture instead of an expected increase in efficacy.

TABLE 5
Loss of Mixed Oxidant Activity (as measured by thiosulfate
titration) for antimicrobial materials.

| | % Loss of Activity at | # Days exposure to mixed |
|---|---|---|
| Antimicrobial | (23° C.) | oxidant |
| DDBSA | 6 | 2 |
| Alkyl Dimethyl amine | 4 | 2 |
| Oxide | | |

In one embodiment of the present invention, an additional antimicrobial composition or agent is added to the mixed oxidant composition to improve efficacy. The additional antimicrobial material can exhibit acceptable stability when exposed to the mixed oxidant for a period of up to 24 hours. Suitable secondary antimicrobial additives may be selected from the group consisting of: chlorhexidine, biguaunide, fatty acids, lactic acid, anionic surfactants, amine oxides, bronopol, amines, nisin, glycerol monolaurate, quaternary ammonium compounds, laurel amine, and combinations thereof. In another preferred embodiment, the secondary antimicrobial additive is dodecylbenzenesulfonic acid (DDBSA). In one embodiment, the secondary antimicrobial additive is alkyl dimethyl amine oxide.

In some embodiments of the present invention, a dairy animal teat sanitizer may be made using one or more concentrates. In one embodiment, a mixed oxidant is mixed with a concentrate plus water to make a solution that can be used on the cow teat to sanitize the teat. The teat sanitizer concentrate will contain from about 500 ppm to about 5,000 ppm mixed oxidant as measured by an iodometric titration using thiosulfate as described above.

A teat sanitizer may be made in a concentrated form for dilution with an aqueous mixed oxidant solution. On a dairy farm, it may be difficult for workers to differentiate between the concentrated and diluted solutions. This may lead to application of concentrate to teats or the dilution of an already diluted solution. In some embodiments, the concentrate contains at least two distinct colorants. The first colorant is one that will degrade and fade when a mixed oxidant is added. The second colorant is not degraded when the mixed oxidant is added. Thus, the concentrate has a first color appearance and the diluted solution ready for use has a second color making it easier for users to safely and properly use the concentrate and ready-to-use teat dip.

The first colorant is preferably selected from the group consisting essentially of: Pigment Violet 23, FD&C Blue 1, Liquitint Blue HP, Liquitint Red ST, Liquitint Pink AL, Yellow #5, Yellow #6, Methylene Blue, FD&C Red #40, Aquamarine Blue, and combinations thereof.

Preferably, the second colorant is a pigmented dye. The second colorant can be, for example, Pigment Green 7, Phthalocyanine Blue BN or Pigment Violet 23, for example.

The stable dairy animal teat sanitizer composition can include phthalocyanine pigment is selected from the group consisting essentially of: Direct Blue 86, Direct Blue 199, C.I. Pigment Blue 15, C.I. Pigment Green 36, C.I. Pigment Green 7, C.I. Pigment Violet 23, and Phthalocyanine Blue BN, and combinations thereof.

It should be understood by those skilled in the art that the terms used herein are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

Also as used utilized in the formulation descriptions herein, the terms “approximately,” “about,” “substantially,” and similar terms are intended to have a broad meaning in harmony with the common and accepted usage by those of ordinary skill in the art to which the subject matter of this disclosure pertains. Further, as used in the formulation descriptions herein, the terms “mixed oxidant” or “mixed oxidants” are intended to include any mixture of oxidants that could be produced during the electrolysis of a metal chloride.

Metal chlorides can include sodium chlorite and potassium chlorite. These oxidants include hypochlorous acid, sodium hypochlorite, potassium hypochlorite, hydrogen peroxide, chlorine dioxide, chlorine and combinations thereof.

For inclusion in the composition of the present invention, the mixed oxidants may be produced using electrolysis or other conventional methods of manufacture. As utilized herein the terms “rheology modifier”, “viscosity modifier” and “thickener” are used interchangeably and are defined to describe materials that can change or affect the rheological or viscosity characteristics of the composition.

Low level sensors 71 and 72 signal the controller 20 through electrical connections 67 and 68 or an operator with an alarm if there is a shortage of an additive within the additive storage tanks 50 and 51. The controller 20 can provide visual and/or audible warnings to an operator so that additive tanks can be re-filled, replaced or provide other attention to the system 100. If an additive is not replaced in a timely fashion, the controller 20 stops the mixing process.

Water is supplied directly from a water tap or an optional water treatment system, including the electrolyzer 19, by opening and closing the water valve 28 using the controller 20. Water 42 passes through the conduit 46 and the meter 92 in controlled quantities. The rate, mass and/or volume of the water flow is controlled by the pressure of the water supply system, conduit size, and/or the water valve 28, and activation of these components by the controller 20. In an alternate embodiment, water can be supplied to the meter 92 using a container and a pump similar to the manner in which the additives are delivered to the mixing manifold 6.

In one embodiment, water is supplied to a reservoir (not illustrated) and then to a pump before passing through the water meter 92. Water level in the reservoir can be controlled in any suitable way, comprising using a float mechanism that activates a water inlet valve until the desired water level is reached. The water in the reservoir can possibly be exposed to microorganisms and may accumulate a biofilm under certain circumstances. These conditions are preferably addressed with chemical additives, cleaners, or a device such as a germicidal lamp or other treatment device placed into or adjacent to the water and internal surfaces of the reservoir. Suitable germicidal lamps are available from UVC LLC, 1780 Bobcat Road, Minden, N.V. 89423 for use in various tanks and containers.

The water meter 92 is connected electronically to the controller 20 through electronic connection 62 or a suitable wireless device. Regardless of how water flow is controlled, water passes through conduit 49 to enter the mixing manifold 6 through inlet 89.

A water quality sensor 21 can monitor water characteristics and transmit water quality data to the controller 20 through the electronic connection 59 or a suitable wireless device. The water quality data can then be used by the controller 20 to determine the quality of the mixed teat dip or to divert the water to a pretreatment system before entering the mixing manifold 6. The water quality data is preferably used to correct or account for the properties of the incoming water as it affects the mixed teat dip properties. Water quality data from the sensor 21 can also be used in comparison to the quality of the final product 9, so that their relative qualities can be considered.

Water and additives are mixed inside the mixing manifold 6 to produce mixed teat dip 9 with a predetermined chemical composition. A static mixer (not illustrated) such as a flow vane, screen or the like, or one or more separate mixing chambers can be incorporated into the mixing manifold 6 (such as at location 94) to accelerate, augment or improve mixing of the fluids. One example of a suitable static mixer is distributed by Stamixco located at 235 84^{th} Street, Brooklyn N.Y. 11209. A heat exchanger can also be used to control the temperature of ingredients in the mixing manifold 6 to aid or control mixing. The mixing manifold 6 is preferably sized to produce substantial mixing of the ingredients for a variety of flow rates. For example, the mixing manifold 6 can be several liters or larger when used in the present invention.

After being mixed, the teat dip product passes through the teat dip quality sensor 22, which is in communication with the controller 20 through electrical connection 69 or a suitable wireless device. Mixed teat dip then passes through the dispense conduit 15 into the storage tank 8 or directly to a dispenser at a dairy milking stall, for example. The storage tank 8 preferably includes an outlet valve 14 for controlling flow of mixed teat dip, and the outlet valve 14 can be operated automatically by the controller 20, manually by an operator, or by an independent means.

The storage tank 8 can include a low level alarm 24 connected to the controller 20 through an electrical connection 76 or a wireless device to notify the controller 20 and/or an operator that the teat dip should be replenished by another mixing operation. The storage tank 8 also preferably includes a full level sensor 23 connected to the controller 20 through an electrical connection 75 or a wireless device that signals the controller 20 when to stop the mixing operation. Starting and stopping the mixing process can be based on other system activities, by manual activation of an operator or by a timer. Mixed teat dip product 9 can also be obtained by an operator from the outlet 10 by manually opening the valve 14.

As stated above, the mixing system 100 of the present invention produces ready-to-use teat dip at a location near where the product is used or stored for a relatively short time (collectively “a point of use”). One advantage of accurately blending water and teat dip chemical additives near the point of use is that local water can be used to make the mixed teat dip. Using water from local sources reduces transportation costs because only the additives need to be packaged and shipped. Further, teat dips that have relatively short shelf lives can be mixed and then used while they are still effective. Another advantage of producing ready to use teat dip at a point of use is that the chemical supplier's plant can reduce capital requirements necessary to mix, package, store, and ship large quantities of premixed teat dip.

Teat dip additives are delivered to a point of use through the normal modes of commerce. Additives used in the present invention can be liquids, but gases and solids (such as powder) can be used by themselves, or in mixtures or suspensions in a carrier. Chemical additives are preferably purchased and delivered directly to a dairy or a dairy chemical dealership in a condition ready to be used by the present invention. Suitable additives include, but are not limited to, antimicrobial agents, liquid surfactants, emollients, viscosity modifiers, fragrances, acids, bases, solvents, alcohols and combinations thereof. The additives can be or can contain water.

In some locations, water that is available at or near the point of use may need to be softened, treated by reverse osmosis or treated by other appropriate methods. The water supplied to the system 100 is preferably pressurized using normal local pressures that are typically between 10 psi and 100 psi. As stated above, the quality of the water can be important, and treatment systems can be employed to change water properties before it enters the mixing system. For example, water softeners reduce the amount of calcium and magnesium in the water, and reverse osmosis reduces unwanted ions from the water.

Also as stated above, the additives are typically not in pressurized tanks, but one or more of the additives can be stored in pressurized containers. When pressurized, additive flow rates can be controlled by a valve or a regulator (not illustrated) that can be opened and closed by the controller 20.

Preferred chemical and water conduits and storage tank materials include stainless steel and plastic. Appropriate filters, check-valves, pressure relief valves and other system components can also be used.

Preferably, the pumps used in the system 100 are self-priming pumps, but they can be any type or configuration comprising positive displacement, rotodynamic pumps that are rotary or reciprocating, or variable speed pumps. The pumps can be controlled in a number of ways, comprising increasing or decreasing the electrical power supply. Preferably, the pumps are operated and sized to deliver ingredients to the mixing manifold 6 at precisely the desired time and ratio to mix the teat dip.

In another embodiment, the additive pumps 82 and 83 are pulsed by activating and deactivating the pumps in rapid succession thereby effectively delivering increased or decreased amounts of a chemical additive to the mixing manifold 6 at a relatively steady rate. This technique produces a result similar to a variable speed pump. A suitable pump for pulsating is a diaphragm pump available from Knight Industries located at 20531 Crescent Bay Drive, Lake Forest, Calif., Model No. EDP 7800.

One or more pumps can have a high pressure trip that allows the pumps to stop operation if line pressure becomes too high. The high pressure trip can inactivate the entire mixing system 100 or sound an alarm.

With the present invention, a precise chemical composition for a teat dip can be mixed in the mixing manifold 6 without additional mixing, by using pumps and valves that are activated and deactivated by the controller 20. One advantage of having a substantially mixed teat dip at the mixing manifold 6 outlet 95 is that the quality of the teat dip can be measured before it is put into the teat dip storage tank 8 or before it is used directly. If the controller 20 determines that teat dip quality does not meet predetermined standards, the teat dip can be diverted so that it is not used, or various types of alarms can alert an operator that quality standards have not been met.

As previously stated, each additive meter 90, 91 is preferably positioned between its respective additive storage container and the mixing manifold 6 to monitor the amount of additive passing to the mixing manifold 6. More preferably, each of the additive meters 90, 91 is positioned between a respective pump and the mixing manifold 6. Alternatively, the meters 90, 91 and 92 can be placed between the additive pumps 82 and 83 and the additive storage tanks 50 and 51. The meters 90, 91 preferably monitor additive mass, volume or flow quantities and send corresponding data signals to the controller 20 corresponding to the quantity of fluid passing into the mixing manifold 6. Preferably, the meters 90, 91, and 92 are oval gear meters (such as those available from Knight Industries, 20531 Crescent Bay Drive, Lake Forest, Calif. Other types of meters can be used in the present invention. Alternately, proportioning valves with feedback loops can be used to control delivery of water and additives in precise quantities. The body and internal components of the meters 90, 91 are preferably manufactured from a chemically resistant plastic or resin and are injection molded. The meter body and oval gears can be manufactured from similar or dissimilar materials.

The meters 90, 91, and 92 can include one or more magnets in monitored spinners to activate a switch to measure the fluid flow. An example of an oval gear meter that can be used in the present invention is described in U.S. Pat. No. 7,523,660 by Albrecht et al. Various oval gear meters, manufactured by Knight Industries located at 20531 Crescent Bay Drive, Lake Forest, Calif., are suitable for the invention.

Preferably, the meters 90, 91, and 92 provide data to the controller 20 for processing, operating the pumps and valves, and, if necessary, adjusting pump and valve operation so that the correct amount of each ingredient reaches the mixing manifold 6 at the proper time. Adjustments can be made as described above by pulsing each pump independently or independently to adjust the output of a variable pump, for example. In this way, precise proportions of ingredients that correspond to the teat dip formula are provided.

The mixed teat dip quality sensor 22 can measure any desired teat dip property such as absorbance, transmittance, density, resistance, impedance, specific gravity, pH, refractive index, conductivity or combinations thereof.

A mixed teat dip quality sensor 22 is preferably employed in the dispense conduit 15 to provide teat dip quality data for recording and ensuring the quality of mixed teat dip 9. The quality data for mixed teat dip 9 can be automatically transmitted by the in-line sensor to the controller 20, so that accurate records for teat dip compositions can be stored and readily available. Additionally, an accurate record of the consumption of total mixed teat dip can be made, stored, and displayed. The teat dip quality and consumption data are preferably stored in any suitable electronic and/or printed form.

Teat dip data from the mixed teat dip quality sensor 22 can be recorded and/or utilized to adjust mixing ratios, to stop production, to activate an alarm, or to divert mixed teat dip that is outside of a predetermined specification away from the storage tank 8 to avoid contaminating previously mixed teat dip.

In one embodiment, the mixed teat dip quality sensor 22 measures the absorbance or transmittance of and a specific wavelength of UV or visible light. This is particularly useful for a dark colored chemical product or a chemical product that contains dye. A dye that is present in the active concentrate can serve as a specific indicator of product quality. The absorbance or transmission of the chemical product at the maximum or other strongly absorbing wavelength of the dye can be measured after the chemicals are mixed in the mixing manifold and a percent of active ingredients can be determined based on an experimentally generated standard curve. Naturally colored materials (such as iodine) containing chemical products can be measured and recorded in a similar manner. Suitable optical sensors are available from Optek located at 45346 Bergeborbeck, Essen, Germany, such as Model OPB733TR.

In another example, the mixed teat dip quality sensor 22 measures specific gravity of the mixed teat dip to determine the concentration of materials like glycerin that are incorporated into the chemical product. A typical specific gravity measuring device is produced by Princo Instruments located at 1020 Industrial Blvd., Southampton, Pa., USA, such as its Densitrol model. Further, the mixed teat dip quality sensor 22 can measure refractive index, which is a good overall indication of proper mixing of various chemicals. In-line refractive index measurement equipment is produced by K-patents located at 1804 Centre Point Circle #106, Naperville, Ill., 60563, USA, Model No. PR23A.

The mixed teat dip quality sensor 22 can also be a conductivity meter or an inductive probe for determining product quality when one of the components of the chemical product has an ionic nature. A conductivity sensor preferably contains a temperature compensation capacity that automatically adjusts the results based on temperatures of the fluids, mixing manifold, or ambient temperatures, for example. In-line conductivity measurement equipment is produced by ASTI located at 603 N. Poplar St., Orange, Calif., USA, Model No. AST50. In another example, the mixed teat dip quality sensor 22 can be a pH meter for determining product quality pH.

In addition to the meters and quality sensors described above, the system 100 can include a temperature sensor or thermocouple at one or more locations to measure the temperature of the additives, water or the mixed teat dip, for example. Temperature probes are often a part of other sensors like conductivity measuring equipment and temperatures can be recorded or otherwise used by the controller 20 as an independent quality measurement.

When the present invention is used in conditions where the ambient temperature changes significantly, ingredients or teat dip properties, such as viscosity, can be affected. Temperature data can be used by the controller 20 to adjust the output of the additive pumps 82 and 83 in relation to additive viscosity and pumping differences of the ingredients when the ambient temperature changes. Flow requirements for the additives can also change when the temperature changes and meter accuracy can be sensitive to changes in temperature and/or viscosity of a fluid. By measuring temperature or viscosity empirically beforehand, any change in flow characteristics determined in the controller 20 can automatically adjust the operation of the additive pumps 82 and 83.

As stated above, the temperature of one or more additives can be controlled with a heat exchanger, for example. This is particularly beneficial when the viscosity and flow characteristics are affected by temperature. Temperature can also be controlled using a heated exterior pad such as those manufactured by Omega Engineering located at One Omega Drive, Stamford, Conn. 06907, Model No. SREH600. The temperature can also be controlled by using an interior heating element that is placed in the container such as those made by F.N. Cuthbert Inc., 3151 South Ave., Toledo, Ohio 43609, Model No. ARMT 2154T1.

In another example of the flexibility of the present invention, the mixed teat dip quality sensor 21 that is installed the conduit 49 can be a conductivity measurement device to measure conductivity of incoming water. Using such a measurement in conjunction with a mixed teat dip quality sensor 22 allows the controller 20 to compensate for any changes in incoming water conductivity that could affect the conductivity of the mixed teat dip product. For example, the controller 20 can use data generated from the water sensor 21 to subtract out the water conductivity from the mixed teat dip conductivity and thereby neutralize the water's conductivity impact on the calculation of the mixed teat dip conductivity. This allows the conductivity reading from the mixed teat dip quality sensor 22 to give a true and accurate reading of the mixed teat dip relative to incoming water.

Similarly, other sensor types can be used to analyze incoming water quality for comparison with mixed teat dip quality. Using the comparison, the controller 20 can automatically change the formulation or simply adjust quality sensor readings to account for variances in water quality. This is particularly important because the source of water used to produce the ready to use teat dip can vary greatly. Local well water or municipal water supply can both be used at a single location, so system flexibility is important from location to location and also at a single location. The amount of ions in the water can vary greatly and if this is not compensated for when conductivity is used as a quality monitor, errors in accuracy of determining the quality of the finished teat dip can occur.

Other sensors that can supply data to the controller 20 about relevant conditions such as; ambient air temperature, incoming water temperature, ambient humidity, or other environmental conditions that affect additives or optimum mixing of teat dip chemicals. Further, ambient conditions can be used to adjust proportions of teat dip ingredients. For example, the proportion of skin conditioners can be automatically adjusted for dry or cold ambient conditions. Other sensors can be used to monitor herd health, and such data sent to the controller 20 can be used for adjusting the type or composition of the teat dip.

The mixing manifold 6 of the present invention is preferably configured to provide substantially complete mixing of all ingredients. As stated above, the mixing manifold 6 can include a static mixing element 94, that is preferably placed between the most downstream additive inlet openings 87 and the sensor 22, so that all of the ingredients are mixed together in the static mixing element 94 before they are sampled by the mixed teat dip quality sensor 22.

Once the proper amount of teat dip has been mixed, data from meters and/or fill gauges 24 is used by the controller 20 to stop all of the flow of all the ingredients at substantially the same time. Nonetheless, various ingredients can be stopped while others continue to flow depending on the mixing tolerances and the ability of the ingredients to mix. As much as plus or minus about 15% of pump or water valve operation time is possible depending on conditions and desired formulations. Mixing teat dip in the mixing manifold 6 eliminates the need for additional mixing after the teat dip exits the mixing manifold 6.

As stated above, the controller 20 can adjust the mixing process depending on conditions. One such condition occurs when one of the additive flow rates through its respective meter is lower than desired. In such a case, the controller 20 receives corresponding data from a pump or meter relating to that ingredient and automatically increases the additive flow rate by adjusting its pump operation and thereby increase that additive's flow rate. Conversely, the flow rates of other ingredients can be reduced to accommodate a flow reduction in any other ingredient.

Preferably, each meter and pump is monitored and controlled independently by the controller 20. In one embodiment, one additive is set to run at a maximum flow rate so that ingredients can be added in different proportions. In another embodiment, the ingredient of greatest proportion (typically water or other carrier) is usually the largest quantity of all components, so that additive flow rates are adjusted by the controller 20 relative to the flow rate of the water. Since the flow rate of the water can vary significantly due to pressure, valve size, system wear, component malfunction or other influences, the adjusting of the flow rates of the additives by the controller 20 allows the mixed teat dip to be made with the proper ratios. Varying water flows can occur within a single production cycle of the chemical product, and immediate adjustments can be made by constantly or intermittently analyzing data from the water meter 21.

The controller 20 can also be used to control the flow rate of one or more of the ingredients by monitoring the quality of the mixed teat dip with the finished product quality sensor 22 or sensors. In one embodiment, the concentration of mixed dip's ionic strength is measured using an in-line conductivity meter or inductive probe. If the ionic strength is outside of a predetermined specification that has been preprogrammed into the controller 20, then the controller 20 increases or decreases the amount of one or more ingredients until the ionic strength is within specifications. If the system cannot be adjusted by the controller 20 to correct a problem and the teat dip does not meet predetermined standards the controller 20 can activate a valve to divert teat dip away from the storage tank 8 so that it is not used on animals.

In another embodiment, the controller 20 increases or decreases the flow rate for one or more ingredients based on a data signal from a temperature sensor. The response of temperature and the flow rate needed for each individual ingredient can be preprogrammed into the controller 20 such that when its temperature increases or decreases, the controller 20 increases or decreases ingredient quantities to maintain the proper ratio of ingredients in the teat dip.

Preferably, the storage container 8 stores teat dip 9 that can be used in normal dairy operations in about one to thirty days. In a preferred embodiment, the storage container 8 is sized to contain enough teat dip for about five to ten days of dairy operation to ensure adequate supply if ingredients are expended and cannot be delivered to the dairy or dairy chemical dealer within several days. If the teat dip contains highly labile ingredients or colorants, the amount of mixed teat dip stored can be less than one day's supply or used immediately to ensure adequate efficacy.

Preferably, after each shift or at the end of the day's work, the storage container 8 is replenished with freshly mixed teat dip. The full level sensor 23 results in substantially the same amount of the teat dip being used each time the system mixes teat dip, but the full level sensor 23 can be adjusted to change the amount of teat dip being mixed. A secondary shut off sensor (not shown) can be installed at a higher location within the storage container 8 in case the primary fill level sensor 23 fails. In a preferred embodiment, an alarm would be activated if the secondary fill level sensor is activated and/or the system 100 will be automatically shut down.

In a preferred embodiment, the mixed teat dip is filled at one level in the storage tank 8 and mixed teat dip 9 is removed for use at a second and different level through conduit 10 by opening the valve 14 to promote a first in first out supply and to minimize storage time for mixed teat dip. The preferred inlet position to fill the storage container 8 is at an upper level in the storage tank 8 and the teat dip is removed from the lower portion of the storage tank 8 for use. This is one way to ensure a first in, first out procedure.

The present invention can mix any number of ingredients and in one example mixes four ingredients such as water, iodine concentrate, emollient, and a thickener to make a low drip teat dip. In another embodiment, four ingredients, such as water, iodine concentrate, emollient, and a solution of poly vinyl pyrrolidone can be mixed to yield a barrier teat dip. Further, one ingredient can be an emollient and a second fluid can be a concentrated iodine solution containing between about 2% to about 10% iodine.

The present invention, therefore, can contain “n” number of meters and “p” number of pumps. When pressurized sources of additives are used in the present invention, then n will be greater than p. If one pressurized source of an ingredient is used in the present invention, that source should preferably be controlled by a valve that is activated by the controller 20. For example, if a pressurized ingredient is entering the mixing manifold 6 at percent volume “x” of the total volume of the pressurized ingredient required for that formulation, then all other ingredients entering the mixing manifold 6 at a rate greater than x of their respective percent volume required for the formulation. The formula for mixing a teat dip can be based on relative volumes of ingredients, relative weights of ingredients or any other property that indicates a proper quantity for the ingredients in any given teat dip. In one embodiment of the present invention, the pressurized ingredient is water, but other carrier fluids can be used in place of or in addition to water. For example, municipal water can be provided through the conduit 42 and the sensor 21 to the mixing manifold 6 at a flow rate determined by the provided water pressure. When the flow rate of the local water source is low, a water reservoir can be installed to provide a suitable supply of water to the system. Alternatively, a surge tank may be placed in line to provide a more even water supply.

Another advantage of the present invention is that it is very compact and occupies a very small footprint. The individual components are easily placed onto a cart or frame that can be transported easily to an end use site. The complete system 100 is low cost because of the efficient use of pumps, meters, and static mixers to provide substantially mixed teat dip that requires no secondary mixing. The present invention can also be manufactured as a module, that can be easily exchanged for an identical module or an upgraded module if a defect or improvement becomes available.

The compact, simple and inexpensive nature of the present invention makes it ideal to be used in a location that is distant from a primary manufacturing location and close to the end use location. Preferably, the system is installed in a dairy facility near the milking parlor. Alternatively, the system can be installed at a dealership location that is closer to the dairy than the primary teat dip manufacturing location. Water that is available at the dairy or dealer is used to make the ready-to-use teat dip to save processing time at the primary teat dip manufacturing location and transportation and packaging costs associated with shipping a premixed ready to use teat dip to a dairy or other remote distant location like a dealer. Mixed teat dip from the invention can be transferred directly into a use conduit if preferred by the user because all teat dip mixed in the present invention is properly ratioed and mixed before leaving the mixing manifold 6. This feature eliminates the requirement of a storage tank or separate mixing feature. While a storage tank or separate mixing feature can be used in certain circumstances, it is not a requirement for mixing quality teat dip using the present invention.

When it is advantageous to change the mixed teat dip formula according to seasonal or environmental conditions at the dairy, a clock or other type of sensor detecting seasonal or environmental changes can be used to send data to the controller 20 to automatically adjust the teat dip formulation. For example, during the summer, a standard low emollient and high iodine teat dip formula can be used, and during the winter a high emollient and lower iodine teat dip can be mixed due to the environmental stress of the cold weather. In one embodiment, this change is activated by a clock function that is synchronized with seasonal changes. In other examples, when unusually cold weather occurs and the system detects this through an ambient temperature sensor, the teat dip formula can be automatically adjusted to a cold weather formula. Other environmental factors that might require specialized formulas include precipitation, humidity or extreme temperatures. In another example, a fly repellant and/or sunscreen is included in the teat dip in response to seasonal and environmental conditions.

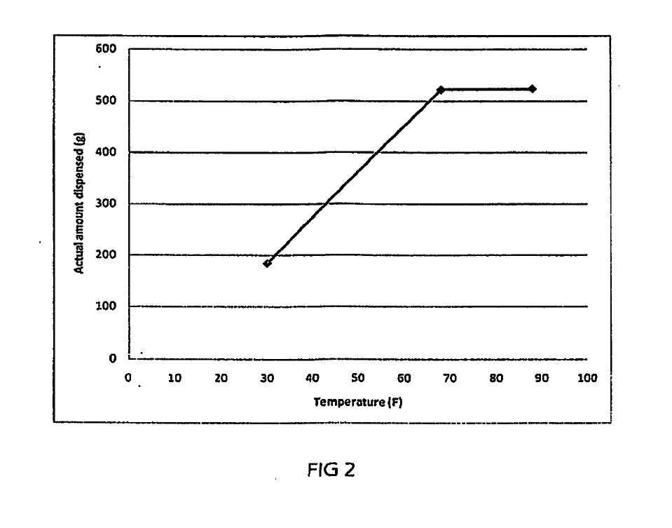

Temperature changes of some teat dip additives as well as system hardware such as, meters, and other system components can change how an additive's flow rate is measured by a meter. In such cases, a temperature response factor can be used in the controller 20 to accommodate such an environmental influence. The graph in FIG. 2 demonstrates how an additive meter could measure iodine concentrate solution in response to a change in temperature. As an example, the graph in FIG. 2 shows that at ambient temperature (68° F.) an additive meter calibrated to 500 ml at 72° F. dispenses 522 g of iodine solution in the same time period. The same meter calibrated the same way might only dispense 184 g of iodine solution at 30° F. This discrepancy can be accounted for using a temperature response factor that is applied to the meter by the controller as a result of determining the temperature of the additive prior to dispensing. The graph in FIG. 2 further illustrates how iodine solution might be dispensed at various temperatures when a meter is calibrated for 500 ml and without the inclusion of a temperature response factor. A temperature sensor can be placed in any or all of the additives to measure the temperature of the additive or additives. Data from the temperature sensor is then provided to controller 20 so that the pump dispensing rates are adjusted accordingly to produce ready to use teat dip that meets specifications.

In another embodiment of the present invention, a teat dip is mixed that is labile, that is, one having constantly changing properties. This occurs when the separate fluids have good long term stability when store separately, but are unstable when combined. This instability prevents teat dip mixing at a location that requires long delivery and storage times before reaching a point of use. An example of this type of a teat dip is a chlorine dioxide teat dip that is preferably mixed at the dairy or nearby dealer because of its short shelf life. For example, within twelve hours to two weeks after mixing, these types of teat dips may no longer be effective and may need to be discarded. In chlorine dioxide teat dip mixtures, one ingredient contains a metal chlorite and another includes an activator for the chlorite. When the ingredients are mixed in the mixing manifold 6 they produce a teat dip that is stable for approximately twelve hours to two weeks. Thus, the system 100 is used to mix no more unstable teat dip than will be used within the stable period of time (“shelf life”).

The controller 20 can also be used to accumulate and store information regarding the frequency, quantity, and quality of teat dip that is produced during a period of time and the type of teat dip produced. Product quality information comprising sensor readings and flow meter readings are accumulated, stored and assigned a specific and unique batch identification number or code in the controller 20 that can be assigned to produce a historical and statistical analysis of the information. The analysis can be downloaded or a user can retrieve the information directly from the controller 20 from a memory card or other removable data storage element, for example. This can be accomplished, for example, by inserting a removable memory device into an appropriate controller port and downloading all or a portion of the information stored within the controller onto the removable memory device. The removable memory device can then be sent to another location and downloaded onto a computer for further analysis and viewing. The transfer of the information and processed information can also be downloaded from the system 100 to a computer using a wireless interface or a cable connection. The internet can also be used to transmit data, preferably over secure lines and/or with encrypted data.

The controller 20 is preferably preprogrammed to perform the following steps:

1. Record beginning and ending meter reading for all meters.
2. Determine temperature of some or all fluids, identify any applicable temperature response factor and apply the response factor to the initial pumping rates if needed at the beginning of each batch production.
3. Preset all pumps to pulse flow at a rate that is predetermined by the expected water flow rate and the formula corrected by temperature response factor for the finished product.
a. Pump pulsing rate for Additive 1=P1
b. Pump pulsing rate for Additive 2=P2
c. Pump pulsing rate for Additive 3=P3
   4. Monitor meters and conductivity sensors every five seconds, continuous is possible.
   5. Compare actual vs. expected flow rates for water and additives based on meter readings.
a. Calculate: (actual water flow/actual additive #1)/(expected water flow rate/expected additive #1)=A1
b. Calculate: (actual water flow/actual additive #2)/(expected water flow rate/expected additive #2)=A2
c. Calculate: (actual water flow/actual additive #3)/(expected water flow rate/expected additive #3)=A3
   6. Correct flow rates of Additives if needed.
a. For A1: If A1>1.05 then increase pump pulsing rate P1 by 5%
b. For A2: If A2>1.05 then increase pump pulsing rate P2 by 5%
c. For A3: If A3>1.05 then increase pump pulsing rate P3 by 5%
d. For A1: If A1<0.95 then decrease pump pulsing rate P1 by 5%
e. For A2: If A2<0.95 then decrease pump pulsing rate P2 by 5%
f. For A3: If A3<0.95 then decrease pump pulsing rate P3 by 5%
   7. Monitor conductivity sensors every five seconds (incoming water and finished product): Store each reading for current Filling
a. Calculate: Corrected Conductivity=(Finished product conductivity)−(Water conductivity) for each 5 second timed sample
b. Evaluate: (Expected Conductivity)/(Actual Conductivity)=C1
   If C1>1.15 or <0.85 then stop system and Activate Alarm
c. Statistics: Perform and record Average, SEM, Range for Incoming Water, Finished Product and Corrected Conductivity C1
   8. Record a unique batch number for every production session that make teat dip.

It should be appreciated that the construction and arrangement of the teat sanitizer, as shown in the various exemplary embodiments, is illustrative only. While the teat sanitizer, according to this invention, has been described in conjunction with the exemplary embodiments outlined above, various alternatives, modifications, variations, improvements, and/or substantial equivalents, whether known or that are or may be presently unforeseen, may become apparent. Accordingly, the exemplary embodiments of the teat sanitizer, according to this invention, as set forth above, are intended to be illustrative, not limiting. Various changes may be made without departing from the spirit and scope of the invention. Therefore, the description provided above is intended to embrace all known or later-developed alternatives, modifications, variations, improvements, and/or substantial equivalents.

1. A dilutable dairy animal teat sanitizer composition comprising:
from about 1% to about 95% of water by weight of composition;
from about 0.5% to about 80% of an antimicrobial agent by weight of composition;
from about 0.05% to about 25% of a thickener by weight of composition; and
from about 0.005% to about 30% of a colorant by weight of composition;
whereby the composition can be diluted with water and a mixed oxidant resulting in a ready to use composition comprising:
from about 1% to about 99% of water by weight of composition;
from about 0.1% to about 80% of the antimicrobial agent by weight of composition
from about 0.01% to about 10% of the thickener by weight of composition;
from about 0.001% to about 10.0% of the colorant by weight of composition; and
from about 0.01% to about 0.8% of the mixed oxidant by weight of composition.

2. The stable dairy animal teat sanitizer composition of claim 1, wherein the composition contains from about 0.1% to about 50% of a stable emollient by weight of composition.

3. The stable dairy animal teat sanitizer composition of claim 2, wherein the stable emollient is selected from a group consisting essentially of:
esters, glycol esters, glycol diesters, PEG esters, amine oxide, and combinations thereof.

4. The dilutable dairy animal teat sanitizer composition of claim 1, wherein the composition is to be diluted with a mixed oxidant at a ratio of about 1:1 composition to diluent to a ratio of about 1:1,000 composition to diluent.

5. The dilutable dairy animal teat sanitizer composition of claim 1, wherein the composition is to be diluted with a mixed oxidant at a ratio of about 1:2 composition to diluent to a ratio of about 1:250 composition to diluent.

6. The dilutable dairy animal teat sanitizer composition of claim 1, wherein the composition is to be diluted with water and a mixed oxidant at a ratio of about 1:5 composition to diluent to a ratio of about 1:50 solution to diluent.

7. The dilutable dairy animal teat sanitizer composition of claim 1, and further comprising a second colorant.

8. The concentrated dairy animal teat sanitizer composition of claim 7, wherein the second colorant comprises Pigment Green 7.

9. The dilutable dairy animal teat sanitizer composition of claim 7, wherein the second colorant comprises Phthalocyanine Blue BN.

10. The dilutable dairy animal teat sanitizer composition of claim 7, wherein the second colorant comprises Pigment Violet 23.

11. The dilutable dairy animal teat sanitizer composition of claim 1, wherein the first colorant is selected from the group consisting essentially of:
Pigment Violet 23, FD&C Blue 1, Liquitint Blue HP, Liquitint Red ST, Liquitint Pink AL, Yellow #5, Yellow #6, Methylene Blue, FD&C Red #40, Aquamarine Blue, and combinations thereof.

12. A concentrated dairy animal teat sanitizer composition comprising:
from about 1% to about 90% of water by weight of composition;
from about 0.1% to about 80.0% of an antimicrobial agent by weight of composition;
from about 0.05% to about 50.0% of a thickener by weight of composition;
from about 0.005% to about 40.0% of a first colorant by weight of composition; and
from about 0.005% to about 40.0% of a second colorant by weight of composition;
wherein the first colorant is degradable in the presence of a mixed oxidant and the second colorant is not degradable in the presence of a mixed oxidant.

13. The concentrated dairy animal teat sanitizer composition of claim 12, wherein the solution is to be diluted with a mixed oxidant at a ratio of about 1:1 composition to diluent to a ratio of about 1:1,000 composition to diluent.

14. The concentrated dairy animal teat sanitizer composition of claim 12, wherein the solution is to be diluted with a mixed oxidant at a ratio of about 1:2 composition to diluent to a ratio of about 1:250 composition to diluent.

15. The concentrated dairy animal teat sanitizer composition of claim 12, wherein the solution is to be diluted with water or a mixed oxidant at a ratio of about 1:5 composition to diluent to a ratio of about 1:50 solution to diluent.

16. The concentrated dairy animal teat sanitizer composition of claim 12, wherein the second colorant comprises Pigment Green 7.

17. The concentrated dairy animal teat sanitizer composition of claim 12, wherein the second colorant comprises Phthalocyanine Blue BN.

18. The concentrated dairy animal teat sanitizer composition of claim 12, wherein the second colorant comprises Pigment Violet 23.

19. The concentrated dairy animal teat sanitizer composition of claim 12, wherein the first colorant is selected from the group consisting essentially of:
Pigment Violet 23, FD&C Blue 1, Liquitint Blue HP, Liquitint Red ST, Liquitint Pink AL, Yellow #5, Yellow #6, Methylene Blue, FD&C Red #40, Aquamarine Blue, and combinations thereof.

20. A stable dairy animal teat sanitizer composition comprised of:
from about 1% to about 99% water by weight of composition;
from about 500 ppm to about 10,000 ppm of a mixed oxidant; and
from about 0.0001% to about 10% of a phthalocyanine pigment by weight of composition.

21. The stable dairy animal teat sanitizer composition of claim 20, wherein the composition contains from about 0.1% to about 50% by weight of a stable emollient.

22. The stable dairy animal teat sanitizer composition of claim 21, wherein the stable emollient is selected from a group consisting essentially of:
esters, glycol esters, glycol diesters, PEG esters, amine oxide, and combinations thereof.

23. The stable dairy animal teat sanitizer composition of claim 20, wherein the phthalocyanine pigment is selected from the group consisting essentially of:
Direct Blue 86, Direct Blue 199, C.I. Pigment Blue 15, C.I. Pigment Green 36, C.I. Pigment Green 7, C.I. Pigment Violet 23, and Phthalocyanine Blue BN, and combinations thereof.

24. The stable dairy animal teat sanitizer composition of claim 20, and further comprising:
from about 0.05% to about 50.0% of thickener by weight of composition.

25. The stable dairy animal teat sanitizer composition of claim 20, and further comprising:
a viscosity modifier is selected from the group consisting essentially of:
PVP, polyvinyl alcohol, polyacrylic acids, carbomers, cross-linked polyacrylic acids, benzene homopolymers, cosolvent polymers, hydrophobically modified copolymers, acrylate/alkyl acrylate cross polymers, polyethylene glycol, and combinations thereof.

26. The stable dairy animal teat sanitizer composition of claim 20, and further comprising:
from about 0.1% to about 80.0% of a secondary antimicrobial agent by weight of composition.

27. The stable dairy animal teat sanitizer composition of claim 26, and further comprising a secondary antimicrobial is selected from a group consisting essentially of:
chlorhexidine, biguaunide, fatty acids, lactic acid, anionic surfactants, amine oxides, bronopol, amines, nisin, glycerol monolaurate, quaternary ammonium compounds, laurel amine, DDBSA, Alkyl Dimethyl amine Oxide, and combinations thereof.
